(19) United States
(12) Plant Patent Application Publication
(10) Pub. No.: US 2003/0172422 P1
(43) Pub. Date: Sep. 11, 2003

(54) Blackberry plant named 'Sonoma'

(76) Inventor: Carlos D. Fear, Aptos, CA (US)

Correspondence Address: PENNIE AND EDMONDS, 1155 AVENUE OF THE AMERICAS, NEW YORK, NY, 100362711

(21) Appl. No.: 09/772,330

(22) Filed: Jan. 29, 2001

(51) Int. Cl. A01H 5/00

(52) U.S. Cl. PLT/203

(57) ABSTRACT

The present invention relates to a new and distinct cultivar of blackberry plant named ‘Sonoma’. The new cultivar is distinguished from other blackberry cultivars by its fruit of excellent fruit flavor and shipping quality. ‘Sonoma’ is a thornless mid-late season cultivar. The new cultivar is distinguished from its seed parent by its larger fruit and greater plant vigor. The new cultivar is distinguished from its pollen parent by its larger, better flavored fruit.

### 1. BACKGROUND OF THE INVENTION

This invention relates to a new cultivar of blackberry called ‘Sonoma’. The new cultivar was developed from hybridization of the patented female cultivar ‘Navaho’, U.S. Plant Pat. No. 6679, with the unpatented male cultivar ‘Hull Thornless’. The parents were crossed in Spring 1991 whereafter fruit and seed were collected to produce seedlings for field planting in Watsonville, Calif. in 1991. The new cultivar was selected in July 1993 for its good flavor, thornless canes, season of production and firm, attractive fruit. The cultivar has been asexually propagated, and reproduced true to type plants by in vitro shoot tip culture.

### 2. SUMMARY OF THE INVENTION

The present invention provides a new and distinct blackberry cultivar named ‘Sonoma’. The variety is botanically identified as Rubus L. subgenus Rubus. The new cultivar produces a floricane crop which begins in early July and continues until mid-September. The new blackberry variety is distinguished from other varieties by a number of characteristics as set forth in Table 1. In particular, the new cultivar is distinguished by its thornless canes with fruit of excellent flavor and firmness which ripens at a time of the year when few other similar cultivars exist.

### 3. COMPARISON TO SIMILAR VARIETIES

The varieties that we believe to be similar to ‘Sonoma’ from those known to us are the male parent ‘Hull Thornless’ and ‘Chester’, both unpatented cultivars. ‘Sonoma’ is particularly different from these cultivars by having slightly larger, more uniform shaped fruit, by ripening earlier, and having a less acidic flavor. Further detailed comparison to ‘Chester’ is presented in Table 1.

### 4. BRIEF DESCRIPTION OF THE DRAWINGS

The accompanying photographs show typical specimens of the fruit, leaves and shoot of the new cultivar, in color as nearly true as reasonably possible in color illustrations of this type.

FIG. 1 is a photograph showing a primocane shoot and mature leaf of ‘Sonoma’.

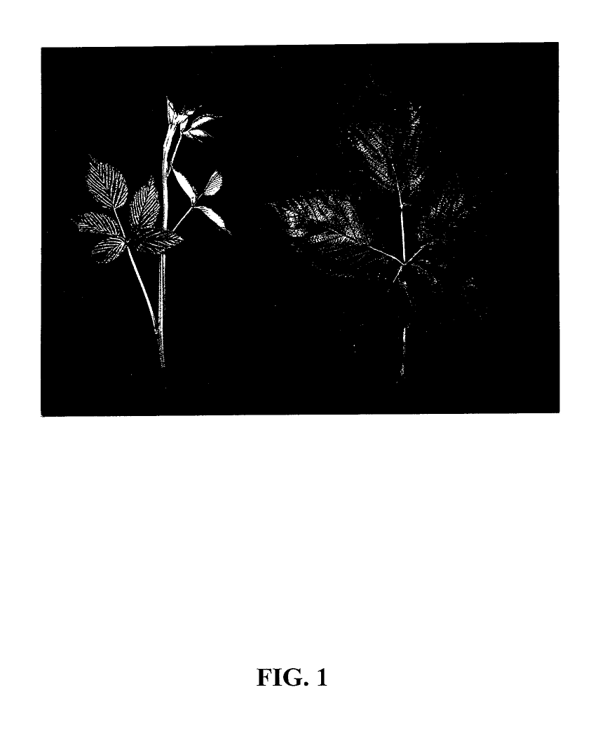

FIG. 2 is a photograph of a ‘Sonoma’ fruiting lateral with fruit in various stages of development.

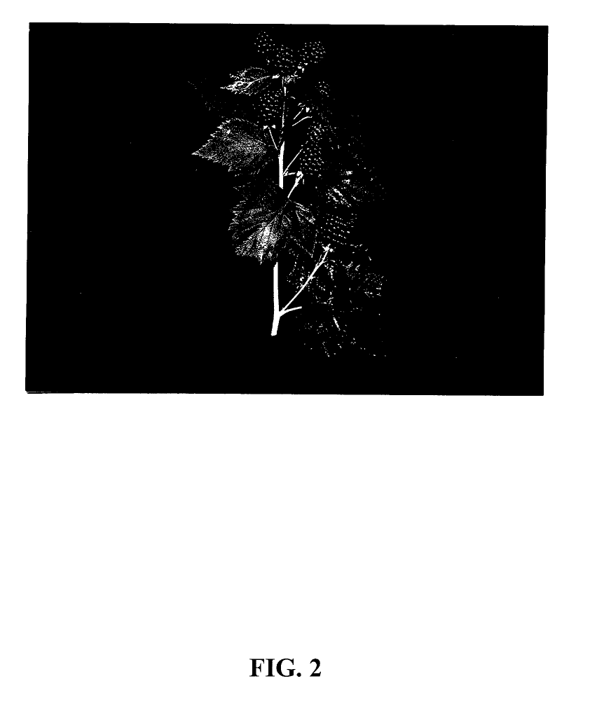

### 5. DESCRIPTION OF THE NEW VARIETY

The following detailed description of the new blackberry cultivar, ‘Sonoma’, is based upon recorded observations of plants and fruit grown between 1996 and 2000 in Watsonville, Calif., and is believed to apply to plants of the ‘Sonoma’ cultivar grown in similar conditions of soil and climate elsewhere. This description is in accordance with terminology used by the International Union for the Protection of New Varieties of Plants (UPOV). Throughout this specification, color names beginning with a small letter signify that the name of the color, as used in common speech, is aptly descriptive. Color data beginning with a capital letter and followed by an alphanumeric code indicate the most similar color designations as provided by The Royal Horticultural Society (RHS) Colour Chart published by the Royal Horticultural Society of London, England. Color designations, color descriptions, and other phenotypical descriptions may deviate from the stated values and descriptions depending upon variation in environmental, seasonal, climatic and cultural conditions.

### 5.1 CHARACTERISTICS OF THE NEW VARIETY

Table 1 provides information on the plant and fruit characteristics of the new blackberry cultivar, ‘Sonoma’, compared with characteristics of the unpatented blackberry cultivars, ‘Olallie’ and ‘Chester’. Both ‘Olallie’ and ‘Chester’ are currently important cultivars for fresh market shipping, and thus are comparable to the proposed use of the new invention, ‘Sonoma’. Observations of ‘Sonoma’ and ‘Chester’ were taken in side-by-side comparison in 1999 and 2000.

The new blackberry cultivar is particularly characterized and distinguished from other cultivars by its fruit with excellent flavor and shipping quality. The fruit of ‘Sonoma’ is very attractive with a solid black color that rarely shows post harvest drupelet color reversion.

The canes of ‘Sonoma’ are thornless and of low to moderate vigor until well established. Yield of the new cultivar is moderate in comparison with other varieties.

Sonoma is distinguished from its pollen parent, ‘Hull Thornless’, by being earlier, less vigorous, and having larger less acidic fruit. Sonoma is distinguished from its seed parent, ‘Navaho’, by having greater vigor and larger fruit.

TABLE 1
PLANT CHARACTERISTICS OF ‘SONOMA’

| | Sonoma | Olallie | Chester |
|---|---|---|---|
| GENERAL | | | |
| Vigor | Low- | Moderate- | high |
| | moderate | high | |
| Growth habit | semi-upright | trailing | semi-upright |
| Productivity | medium | high | high |
| Self fruitfulness | yes | yes | yes |
| Number of young shoots | medium | medium | medium |
| CANES | | | |
| Primocanes | | | |
| Anthocyanin coloration | absent | present | present |
| Spines | absent | present | absent |
| color | — | purple | — |
| attitude of tip | — | horizontal | — |
| texture | — | heavy | — |
| presence and distribution | absent | present; | absent |
| on petioles | | irregularly | |
| | | distributed | |
| density in central | — | medium | — |
| third of shoot | | | |
| Internodal distance (cm) - | 3 | 2.6 | 3.1 |
| central third | | | |
| of mature cane | | | |
| Glaucosity on full | weak | weak | weak |
| grown shoot | | | |
| Strength of full | strong | medium | strong |
| grown shoot | | | |
| Cane cross section | angular | rounded to | angular to |
| | | angular | grooved |
| LEAVES | | | |
| Relief between veins | medium | medium | medium |
| Number of leaflets | usually 5 | usually 3 | usually 5 |
| Leaf color | medium | medium | light |
| upper side | 139A, 147A | 137A, 137B | 147A |
| underside | 147B | 147B | 146A |
| Glossiness of upper surface | medium | medium | dull |
| Leaf cross section | concave | concave-flat | concave |
| Terminal leaflet | | | |
| length (cm) | 10.8 | 8.9 | 11.1 |
| width (cm) | 8.5 | 7.6 | 9 |
| shape | cordate | cordate | cordate |
| tip | acuminate | acuminate | acuminate |
| base | rounded | cordate | cordate |
| margin | double | double | double |
| | serrate | serrate | serrate |
| Lateral leaflet | | | |
| overlap of lateral leaflets | overlapping | overlapping | overlapping |
| length (cm) | 10.3 | 8.7 | 10.2 |
| width (cm) | 7.1 | 6.1 | 7.1 |
| shape | ovate | ovate | ovate |
| tip | acuminate | acuminate | acute |
| base | rounded | acute | acute |
| | to acute | | |
| margin | double | double | serrate |
| | serrate | serrate | |
| Petiole | | | |
| mean length (cm) | 9.4 | 5.3 | 7.9 |
| range | 7.3-11.1 | 3.6-8.7 | 3.9-10.2 |
| pigmentation of | reddish | green- | purple |
| upper surface | | slightly | |
| | | pink | |
| pigmentation of | green | green- | green- |
| underside | | slightly | pinkish |
| | | pink | |
| Length of stalklet | short | very short | medium |
| Rachis length (cm) between | 3.8 | 2.8 | 3.1 |
| terminal and adjacent | | | |
| lateral leaflets) | | | |
| Stipule orientation | erect | variable; | erect |
| | | clasping | |
| | | to erect | |
| FLOWERS | | | |
| Time of bud burst | late | early | late |
| Time of beginning | late | early | late |
| of flowering | | | |
| Flower size | medium- | small to | small to |
| | large | medium | medium |
| Petal size | | | |
| length (mm) | 19.6 | 16.5 | 18.3 |
| width (mm) | 14 | 11.7 | 10.9 |
| Anthocyanin color | absent | absent | present |
| of pedicel | | | |
| Intensity of pedicel | — | — | weak |
| coloration | | | |
| Length of pedicel | short | long | short |
| Flower number (third | 1.25 | 3.6 | 2 |
| node from tip of lateral) | | | |
| FRUIT | | | |
| Harvest season | mid-late | early | mid-late |
| Dimensions | | | |
| weight (g/fruit) | 3.6 | 5.2 | 3.2 |
| size | medium- | medium | small |
| | small | | |
| length (cm) | 2.4 | 3.3 | 1.9 |
| width (cm) | 2.1 | 1.4 | 1.9 |
| Fruiting lateral length | medium- | medium | medium- |
| (in mid cane) | long | | long |
| mean number of fruit | 11.6 | 6.2 | 22.8 |
| per lateral | | | |
| range | 8-16 | 3-9 | 17-40 |
| Shape | ovate to | narrow ovate | round to |
| | elliptic | much longer | ovate as |
| | longer than | than | long as |
| | broad | broad | broad |
| Color | black | purple- | black |
| | | black to | |
| | | black | |
| immature | 183A | 178A-183B | 184A |
| maturing | 187A | 187A | 200A-202A |
| mature | 202A | 200A | 202A |
| Firmness | medium | medium | firm |
| Glossiness | medium | medium- | medium |
| | | strong | |
| Soluble solids | 12 | 9.7 | 9.9 |
| Titratable acidity (% as | 9 | 13.3 | 9.9 |
| citric acid) (ml of added | | | |
| 0.1 N NaOH to pH 8.1) | | | |
| Number of drupelets | 60 | 86 | 40 |
| per fruit | | | |

### 5.2 NUCLEIC ACID FINGERPRINTING

Distinctive patterns of polymorphism can be detected using a variety of nucleic acid analysis methods. In one non-limiting example, molecular genetic maps can be produced using random amplified polymorphic DNA (RAPD) (Williams et al., 1990, “DNA polymorphisms amplified by arbitrary primers are useful as genetic markers”, Nucleic Acids Res. 18(22):6531-5). Using a variety of oligonucleotide primers, alone or in combination, RAPD analysis of Sonoma, Chester, and Olallie yielded DNA fragment patterns that uniquely distinguish each of these genetically distinct genotypes.

We claim:

1. A new and distinctive cultivar of blackberry plant, substantially as shown and described.
